(19) United States
(12) Patent Application Publication
(10) Pub. No.: US 2014/0336147 A1
(43) Pub. Date: Nov. 13, 2014

(54) HEMOSTATIC AGENTS AND METHODS OF USE

(71) Applicant: AGRATECH INTERNATIONAL, INC., Kinnelon, NJ (US)

(72) Inventors: Dean A. Berman, Kinnelon, NJ (US); Joseph F. Bristow, Lafayette, LA (US)

(21) Appl. No.: 14/364,463

(22) PCT Filed: Dec. 12, 2012
(86) PCT No.: PCT/US2012/069134

§ 371 Date: Jun. 11, 2014

Related U.S. Application Data
(60) Provisional application No. 61/569,572, filed on Dec. 12, 2011.

(51) Int. Cl. A61K 9/107; A61K 9/00; A61K 31/722

(52) U.S. Cl. CPC A61K 9/107; A61K 31/722; A61K 9/0014; A61K 9/0017; A61K 9/0043
USPC 514/54

(57) ABSTRACT

Modified chitosan hemostatic agents include reverse micelles (10) having an outer hydrophobic shell (12) of suitable biocompatible hydrophobic components such as alkanes, and hydrophilic positively charged chitosan moieties (14) enclosed within the hydrophobic shell (12). The hydrophobic shell (12) may be formed by attaching hydrophobic moieties to one end of chitosan polymer molecules while retaining sufficient reactive (positively charged) amine groups on the remainder of the chitosan polymers for effective blood clotting. The resulting reverse micelles (10) have the capability of penetrating mucous membranes such as nasal mucosa (20) or otherwise penetrating to bleed sites shielded or partially shielded by mucosa or other tissues. Upon exposure to red blood cells (24) escaping from the bleed site (26) the positively charged interior of the reverse micelles breaks through the hydrophobic shell (12) to combine with the negatively charged red blood cells (24) thereby clotting and attenuating or stopping the bleeding.

### CROSS-REFERENCE TO RELATED APPLICATION

This application claims the benefit of priority of provisional patent application Ser. No. 61/569,572, filed on Dec. 12, 2011, entitled “Hemostatic Agents and Methods of Use”.

### FIELD OF THE INVENTION

The present invention generally relates to modified chitosan agents capable of staunching bleeding at sites which are difficult to access, or at sites which are shielded, such as sub-mucosa and other shielded-sites, and to methods of using such agents.

### RELATED ART

U.S. Published Patent Application U.S. 2009/0062849 A1 of Matthew Dowling et al., published on Mar. 5, 2009, discloses a hemostatic tissue sealant sponge and a hemostatic tissue sealant spray for staunching bleeding from acute wounds. One disclosed sponge embodiment utilizes a biopolymer such as chitosan which has been hydrophobically modified to provide a plurality of short hydrophobic substituents attached to the backbone of the chitosan polymer. Hydrophobic components extending from the chitosan backbone interact with the bi-layer membrane of tissues or cells and is said to provide a seal which is strong enough to contain blood within the boundaries of the sponge, yet weak enough to substantially prevent damage to newly formed tissue upon removal of the sponge.

Generally, the hemostatic or blood-clotting properties of chitosan are well known. Chitosan has been used in bandages and, as suggested by the Dowling et al. Application, a spray, for direct application to open wounds. Chitosan is also known for use in gels or foams to be directly applied to open wounds to stop or control bleeding until a patient can receive more effective medical procedures to staunch bleeding, such as cauterizing or stitching wounds.

Chitosan nasal gel formulations are also known for use in the treatment of colds and flu.

Reverse micelles are known in the art. For example, Daedalus Innovations, LLC of Philadelphia, Pa. employs reverse micelles for encapsulation of macromolecules, for the purpose of conducting structural studies employing nuclear magnetic resonance (“NMR”) spectroscopic techniques.

### SUMMARY OF THE INVENTION

In accordance with the present invention there are provided hemostatic agents comprising a chitosan polymer having hydrophobic moieties attached thereto, the hydrophobic moieties being biocompatible, and of a kind and present in quantities at least sufficient to form a reverse micelle. A reverse micelle is a micelle wherein the exterior of the shell is hydrophobic as it is comprised of hydrophobic groups, such as alkanes or other suitable hydrocarbon compounds, attached at or near one end of the chitosan polymer chains. The hydrophobic shell is non-ionized. The interior of the shell is hydrophilic and is comprised of positively charged chitosan moieties, i.e., portions of the chitosan chains remote from the hydrophobic moieties and having reactive sites.

One aspect of the present invention concerns hemostatic agents comprising hydrophobically-modified chitosan (“HMC”) reverse micelles which are able to penetrate mucous membranes and then release or expose positively charged chitosan moieties to react with red blood cells escaping from the bleed site, to thereby effectuate clotting of the blood. Hemostatic agents of the present invention are capable of penetrating mucosa in an osmosis-like manner without damaging the mucosa and then forming clots to staunch the bleeding at the bleed site, for example, from ruptured capillaries or the like.

Generally, the present invention is applicable not only to wounds or other bleeds sustained by humans, but also has veterinary uses to staunch bleeding in wounds or other bleeds of animals. The present invention enables delivery of the chitosan to the bleed site itself, even if that bleed site is fully or partially shielded by mucosa or by other tissues or organs of the patient being treated. The chitosan will, of course, also be effective at locations somewhat removed from the bleed site itself, for example, on the surface of the mucosa as blood from the bleed site seeps through the mucosa. Other specific uses of the hemostatic agent of the present invention are to treat, in addition to nose bleeds, post-operative bleeds such as tonsil bleeds, intracranial bleeds, gastric bleeds, rectal bleeds, gastrointestinal bleeds, urinary tract bleeds, pulmonary bleeds, cardiovascular bleeds, tissue bleeds generally, eye bleeds and ear bleeds. Such bleeds may be endogenous or may be post-operative or may occur as a result of an accident or other physical trauma.

In another aspect of the present invention, the hemostatic agents are delivered to bleed sites by any suitable delivery means or mechanism.

In one aspect of the present invention, the hemostatic agents are applied in the form of a spray, such as a nasal spray, but any delivery mechanism suitable for the bleeding site or sites involved may be employed. For example, the hemostatic agents of the present invention may be applied enterally, e.g., orally, rectally or sublingually, parenterally, e.g., intravenously, intramuscularly, or subcutaneously, or by other methods such as inhalation, e.g., by nebulization for application to the lungs, topically or transdermally. The hemostatic agents may be in any suitable physical form, such as foams, gels, sprays, fine particulate solids, liquid suspensions, etc.

In other aspects of the present invention the hemostatic agent may comprise, instead of a reverse micelle, a conventional micelle having a hydrophilic chitosan external shell and a hydrophobic interior.

The present invention finds application in staunching bleeding generally, but it is particularly efficacious in staunching bleeding from other than external, easily accessible wounds, that is, from any bleed site which is shielded or partially shielded by mucosa and/or epithelium, sub-mucosa or any capillary, arterial or venous bleed site. The present invention provides for delivery of hemostatic agents to internal and/or shielded bleed sites and therefore finds application in staunching bleeding resulting from surgery, for example, from oral, ear, nose or throat surgery, and in staunching dermatological, gastrointestinal, pulmonary, etc., bleed sites.

Specifically, one aspect of the present invention provides a hemostatic agent comprising hydrophobically modified chitosan (“HMC”) reverse micelles comprised of chitosan polymer molecules having attached thereto hydrophobic biocompatible moieties of a kind and present in an amount sufficient to convert the chitosan molecules into the HMC reverse micelles, the micelles having a hydrophobic exterior provided by the hydrophobic moieties and a hydrophilic interior comprised of positively charged chitosan moieties.

A method aspect of the present invention provides for staunching bleeding in humans or animals, the method comprising delivering to a bleed site or to the vicinity of a bleed site hydrophobically modified chitosan (“HMC”) reverse micelles, the HMC reverse micelles comprising chitosan polymer molecules having attached thereto biocompatible hydrophobic moieties of a kind, and present in an amount at least sufficient, to form the HMC reverse micelles having a hydrophobic exterior provided by the hydrophobic moieties and a hydrophilic interior comprised of positively charged chitosan moieties.

Another method aspect of the present invention provides for staunching bleeding in humans or animals by delivering to a bleed site or to the vicinity of a bleed site hydrophobically modified chitosan (“HMC”) reverse micelles comprising chitosan polymer molecules having attached thereto biocompatible hydrophobic moieties of a kind, and present in an amount at least sufficient, to form the HMC reverse micelles having a hydrophobic exterior provided by the hydrophobic moieties and a hydrophilic interior comprised of positively charged chitosan moieties, the delivery being accomplished enterally, parenterally, by inhalation or topically.

Other aspects of the present invention are disclosed in the following description and the appended drawings.

### BRIEF DESCRIPTION OF THE DRAWINGS

FIG. 1 is a schematic cross-sectional view of a generally spherical hydrophobically-modified chitosan reverse micelle in a non-polar solvent, in accordance with one embodiment of the present invention;

FIG. 2 is a schematic cross-sectional illustration of a mucous membrane overlying a capillary bed and illustrating penetration of the mucous membrane by the reverse micelle of FIG. 1 and its action in staunching bleeding from one or more bleed sites on the capillaries;

FIG. 3 is a schematic cross-sectional view corresponding to that of FIG. 1 but showing the outer shell of the reverse micelle being broken and penetrated by the positively charged hydrophilic chitosan moieties; and

FIG. 4 is a conventional micelle utilizable in certain aspects of the present invention and having a hydrophilic chitosan exterior shell enclosing hydrophobic components comprised of hydrophobic molecules attached at or near one end of the chitosan chains.

### DETAILED DESCRIPTION OF THE INVENTION AND SPECIFIC EMBODIMENTS THEREOF

As indicated above, it is known to use chitosan, including hydrophobically modified chitosan, for direct application to accessible open wounds in order to staunch bleeding.

However, there are numerous situations in which a patient is suffering bleeding from one or more sites which may be fully or partially “shielded” from direct external applications of hemostatic agents. Such shielded sites may be internal sites and/or may be sites covered by mucous membranes (“mucosa”). Sub-mucosa bleeding sites are, for example, rectal bleeding as from hemorrhoids, gastric bleeding as from ulcers or the like, pulmonary (lung) bleeding, oral bleeding, intraocular bleeding, nasal bleeding, etc. Other internal bleed sites are intraarterial sites such as an aneurism, intraarticular sites such as post-trauma bleeding in a knee or elbow joint, etc. One such common situation among many others is epistaxis, i.e., nosebleeds. In both anterior nosebleeds, which make up about 90 percent of nosebleed cases, and posterior nosebleeds, the bleeding is through capillaries located in tissue which is below the patient's mucous and basement membranes.

Current treatment modalities for epistaxis include:

1) Direct pressure, i.e., pinching the nose. This is a poor technique that does not always work and is difficult for some patients, especially the elderly.

2) Nasal packing with gauze, nasal tampon or other device. These are painful, require antibiotics to prevent toxic shock and create a physical obstruction to air flow.

3) Local application of vasoconstrictors such as oxymetazoline or phenylephrine. These may increase the patient's blood pressure and, inasmuch as many patients with epistaxis already have high blood pressure, vasoconstrictors may be too risky to use. Vasoconstrictors may also cause rebound nasal congestion if used chronically.

4) Products such as those sold under the trademarks Surgicel and Gelfoam promote clotting (coagulation) of blood. As these products must be placed in the nasal cavity of the nose, they have the same disadvantages as nasal packing and, in addition, are relatively expensive.

5) Chemical cautery with silver nitrate. This treatment is painful and may perforate the septum. Further, it is sometimes difficult to locate the exact vessel from which bleeding is occurring and onto which the silver nitrate must be applied.

A chitosan-based nasal spray or other delivery system which is able to penetrate mucosa and deliver to the bleed site the hydrophobically modified chitosan of the present invention will halt nose bleeds without the need for packing or the other modalities noted above. Chitosan is biodegradable, hypoallergenic and non-toxic, minimizing risk and discomfort for the patient. One embodiment of the present invention provides a spray formulation which will penetrate nasal tissue and reach the ruptured capillaries, forming clots at the source of the bleeding. The spray may be contained in a plastic spray bottle typical of those used for other nasal sprays and introduced into one or both nostrils of a patient experiencing nasal bleeding.

In another application, reverse micelles containing chitosan may be injected intraarterially just proximal to a bleeding site in order to form a clot or “plug” at the opening of a capillary or artery to stop bleeding. A hydrophobic “bubble” may be incorporated into the wall of a leaking vessel, so that positively charged chitosan is released to form a clot in the sub-mucosa and endothelial lining of the vessel. A suitable shield or “screen” may be emplaced immediately downstream of the site in order to prevent any downstream migration of the reverse micelles or the resulting clot. Clinical uses of the hemostatic agent of the invention include but are not limited to: 1) neurosurgery to stop a bleeding aneurism, or parenchymal bleed instead of or in addition to currently used coils, 2) to staunch retinal bleeds/hemorrhages, 3) to staunch diverticular bleeding, 4) to staunch aortic aneurisms, 5) to staunch bleeding from esophageal or gastric varices, 5) to staunch intraarticular bleeds, i.e., to stop post-trauma bleeding in knee or elbow joints by sealing the joint surface and forming a plug where needed.

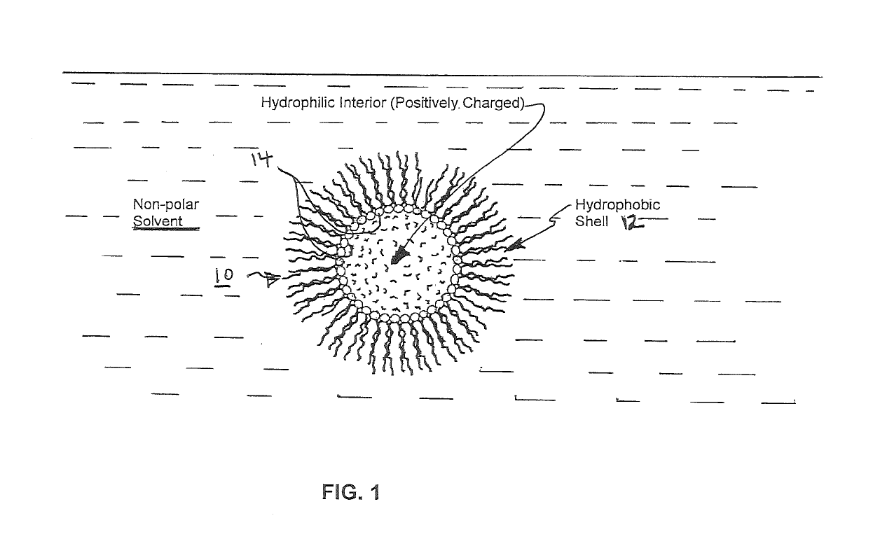

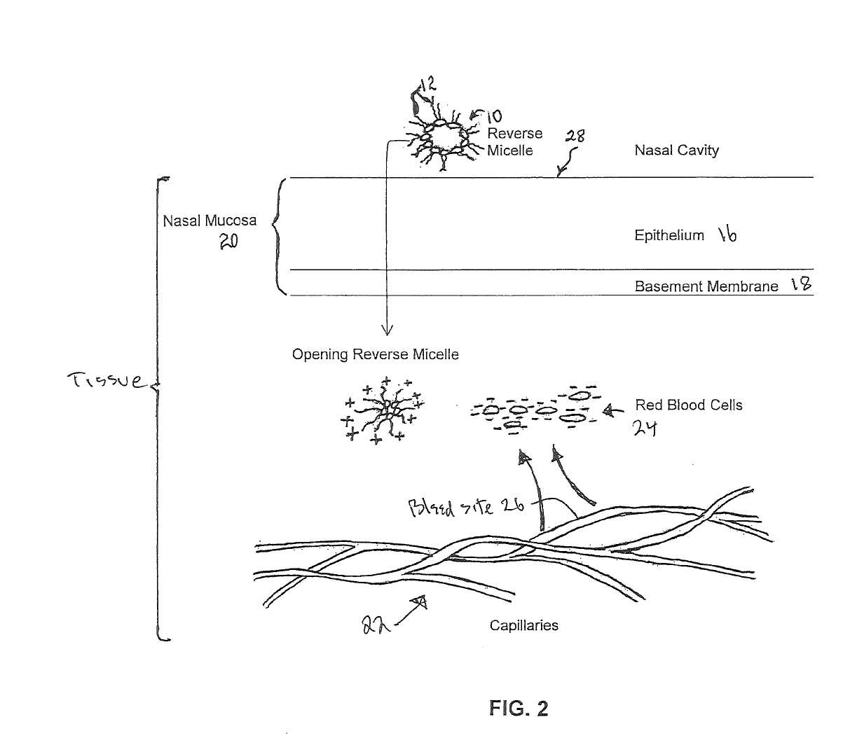

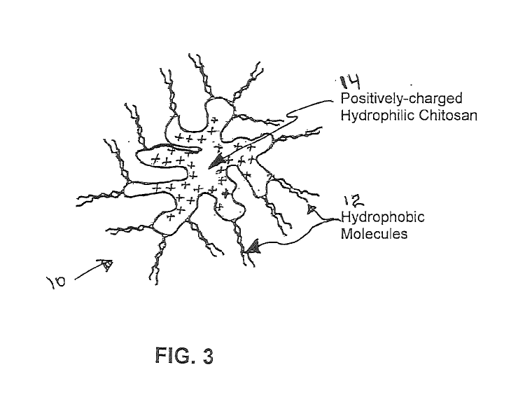

Generally, hemostatic agents of the present invention, including the nasal spray embodiment, may comprise a non-polar solvent containing hydrophobically modified chitosan in a reverse micelle configuration as schematically illustrated in FIG. 1. The reverse micelle 10 comprises a hydrophobic shell 12 with hydrophilic chitosan moieties 14 inside. As shown in FIG. 2, the hydrophobic shell 12 allows the reverse micelle 10 to penetrate the epithelium 16 and basement membrane 18 of the nasal mucosa 20 to reach the capillary plexus 22 from which red blood cells (“RBCs”) 24 are leaking from the bleed site 26. In the aqueous environment of the tissue and in the presence of RBCs 24, which are negatively charged, the reverse micelle 10 breaks down (FIG. 3) as the positively-charged chitosan moieties 14 are attracted to, and bond with, the negatively-charged RBCs 24 (FIG. 2), thereby forming clots. It will of course be appreciated that in any given case, a large quantity of reverse micelles, perhaps millions, will be supplied to the bleed site.

The reverse micelle 10 breaks down because of the strong attraction between the positively charged chitosan moieties 14 and the negatively charged red blood cells 24 which exist in profusion at the bleed site.

Referring to FIG. 2, blood which has seeped through the nasal cavity surface 28 of the epithelium 16 will similarly cause reverse micelles 10 to break down at the surface 28 to aid in staunching bleeding. A great advantage of the nasal spray embodiment of the invention (and other embodiments) is the ability to penetrate mucosa (20 in FIG. 2) to get to the bleed site (26 in FIG. 2) to clot the blood at the source of the bleed.

Charged chitosan might cross the capillaries also. There may be cases where the mucosa or epithelium is “worn” down so much as to expose the capillaries close to the mucosa surface.

As is well understood in the art, chitosan is chitin which has undergone at least 40% deacetylazation, i.e., at least 40% of the chitin's acetyl groups have been removed and replaced with other moieties, typically, amine groups, by methods known in the art. The chitosan may have any suitable degree of deacetylation, e.g., 50%, 60%, 70%, 80%, 90% or more, e.g., 95%. See U.S. Patent Application Publication No. U.S. 2009/0275745 A1 of Joseph Bristow, published Nov. 5, 2009 and entitled “Chitosan Manufacturing Process”, which issued as U.S. Pat. No. 8,318,913 on Nov. 27, 2012, the entire disclosure of which is incorporated by reference herein. Chitin is a biopolymer which is obtained from a variety of sources, commonly from the shells of crustaceans. Shrimp shells are a major source of chitin.

The chitosan used in the nasal spray formulation embodiment of the present invention preferably has a degree of deacetylation greater than 90% in order to provide ample reaction sites for the hydrophobic molecules as well as unreacted sites that can protonate for red blood cell attraction. The molecular weight of the chitosan for the nasal spray application preferably is no greater than 60,000 Daltons (60 kDa) to keep the viscosity of the solution low enough to be sprayed while maintaining a large molecular chain length for adequate clot formation. Very low molecular weights of chitosan may be used for spray applications, for example, from about 5,000 Daltons up to not more than about 60,000 Daltons. For example, a chitosan molecular weight of from about 5,000 to 30,000, 5,000 to 50,000, 10,000 to 60,000 or 10,000 to 50,000 Daltons. Higher molecular weights of chitosan may be employed for non-spray applications, the upper limit on the molecular weight being determined by the desired mobility of the reverse micelles to penetrate tissue in order to access “shielded” (by tissues or organs) bleed sites. An intermediate level of molecular weights would be selected based on the desired penetration of the reverse micelles into tissue or organs in a given case or application. For use in surface bleed sites, chitosan of high molecular weight may be used, e.g., up to about 2,000 KDa, for example from greater than 60 KDa, e.g., about 61 KDa to about 500 KDa, or from about 65 KDa to about 1,000 KDa.

To create the reverse micelle, for example, to form the nasal spray, chitosan is first modified in a mild acetic acid solution to attach to the chitosan a hydrophobic molecule of sufficient size to create the reverse micelle 10 in FIG. 1). The solution is then mixed in a non-polar solvent and agitated. The reverse micelle is formed in the non-polar solvent by the hydrophobic molecules forming an outer “shell,” while the unmodified hydrophilic portions of the chitosan polymer molecule, which are insoluble in the non-polar solvent, congregate inside the hydrophobic shell. The concentration of hydrophobic molecules on the chitosan polymer molecule, and the size of the hydrophobic molecules, are controlled such that there is sufficient attraction of the hydrophobic molecules to the non-polar solvent to form the reverse micelles, while leaving sufficient protonated amine groups on the chitosan polymer molecule available to attract RBCs. The non-polar solvent containing the modified chitosan is separated from the mild acetic acid solution and packaged in a nasal spray bottle.

The ability to penetrate mucosa provides an important advantage over prior art topical applications of chitosan which lack such penetrating ability. Such topical applications encounter the bleeding only on the surface of the patient's body, that is, clotting of the blood starts from the outer surface of the wound. While the chitosan may build a barrier, that barrier may fail due to the pressure and flow rate of the bleeding.

The positively-charged chitosan moiety and the attached hydrophobic molecules are free to equilibriate back across the mucosa in the reverse direction to transfer the chitosan moiety to the mucosa surface for additional clot formation. Hemostatis may thus be effectuated both at a sub-mucosa or other tissue bleed site and at the surface. Generally, without wishing to be bound by any theory, it is believed that the exterior hydrophobic envelope or bubble aids in absorption of the reverse micelles while encasing and protecting the positively charged chitosan particles during transportation through the mucosa or other tissue. Preferably, the hydrophobic molecules are selected to provide an electrically neutral, that is, non-ionized, protective outer shell in order to provide better absorption through the mucosa or other tissues.

Any one of a large number of hydrophobic molecules which are biocompatible, that is, which may be safely introduced into a human or animal body, may be employed. These include alkanes, amino acids, or any suitable biocompatible hydrophobic molecules. As used herein, the term “biocompatible” has its usual meaning of being suitable for introduction into a living person or animal without doing unacceptable or any harm. In addition to alkanes, other suitable hydrophobic molecules may be biocompatible compounds as follows. Carbon compounds of the formula CxAy wherein C is carbon, A is selected from hydrogen, oxygen, nitrogen and x and y are integers; As noted above, suitable amino acids, such as H₂NCH₂COOH, may be used.

Without wishing to be bound thereby, the following are believed to be possible mechanisms of diffusion of the reverse micelle.
a) The reverse micelles just diffuse through the lipid membrane, charged chitosan is released below the basement membrane and is also able to move into capillaries to stop bleeding prior to the blood reaching the mucosa.
b) The reverse micelles follow a break in mucosa, charged chitosan is gradually released at the mucosa and sub-mucosa and then below the basement membrane into the capillary plexus.
c) Positively charged chitosan is released where the greatest concentration of negatively charged red blood cells are. Equilibrium could be obtained when a membrane is involved. In addition to lipid diffusion, other potential mechanisms are aqueous diffusion, diffusion by special carriers and pinocytosis (receptor-mediation endocytosis).

A reverse (or inverse) micelle has the positively charged chitosan (ionized) on the inside of a hydrophobic (non-ionized) outside bubble.

The highly charged chitosan, once it crosses the basement membrane of the mucosa will be able to interact with the negatively charged red blood cells that are leaking from the vascular capillary plexus to form a clot and thus cause hemostasis. Without wishing to be bound by any particular theory, it is believed that the chitosan reverse micelle may cross membranes or other barriers by four potential mechanisms: aqueous diffusion, lipid diffusion, via special carriers (facilitated diffusion), and/or by pinocytosis (receptor-mediated endocytosis).

For most mechanisms of permeation the rate of diffusion (magnitude of flux) is determined by Fick's Law of Diffusion. In the case of a passive process, the concentration gradient driving it is important.

The hydrophobic bubble would allow transfer of the reverse micelle through the lipid membrane. Once the reverse micelle travels past the basement membrane the concentration of positive ions inside of the bubble will be so high that it crosses the bubble to attach to negatively charged red blood cells.

Once equilibrium was met, the reverse micelle would stop releasing the positively charged chitosan below the basement membrane because the red blood cells would be saturated with positively charged ions at which point the concentration of positively charged chitosan below the basement membrane would be too high for further release of positively charged chitosan. That condition would allow some of the reverse micelles to traverse the basement membrane in the opposite direction to allow the reverse micelles to settle near the surface mucosa of the epithelium where the positively charged chitosan can diffuse passively towards the surface of negatively charged red blood cells.

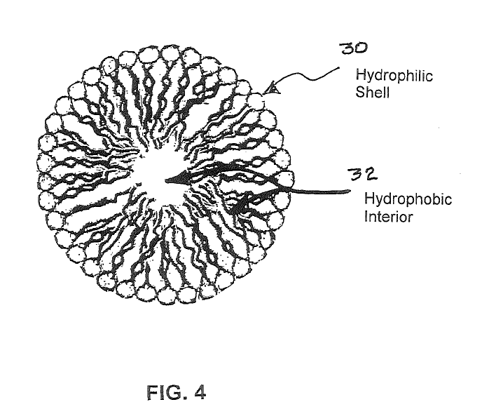

FIG. 4 shows a normal, that is, a non-reverse, micelle having a hydrophilic shell 30 and a hydrophobic interior 32. Shell 30 may comprise the positively charged chitosan moieties and hydrophobic interior 32 may comprise hydrocarbon compounds such as alkanes.

While the invention has been described in detail with respect to specific embodiments thereof, it will be appreciated that numerous other embodiments lie within the scope of the present invention and the appended claims.

What is claimed is:

1. A hemostatic agent comprises hydrophobically modified chitosan (“HMC”) reverse micelles comprised of chitosan polymer molecules having attached thereto hydrophobic biocompatible moieties of a kind and present in an amount sufficient to convert the chitosan molecules into the HMC reverse micelles, the micelles having a hydrophobic exterior provided by the hydrophobic moieties and a hydrophilic interior comprised of positively charged chitosan moieties.

2. The hemostatic agent of claim 1, wherein the hydrophobic moieties are selected from the class consisting of alkanes and amino acids.

3. The hemostatic agent of claim 1, wherein the hydrophobic moieties are attached to one end of respective chitosan polymer molecules leaving the other end of such molecules to serve as the positively charged chitosan moieties.

4. The hemostatic agent of claim 1 in a physical form selected from the group consisting of foams, gels, sprays, fine particulate solids and liquid suspensions.

5. A method of staunching bleeding in humans or animals comprising delivering to a bleed site or to the vicinity of a bleed site hydrophobically modified chitosan (“HMC”) reverse micelles, the HMC reverse micelles comprising chitosan polymer molecules having attached thereto biocompatible hydrophobic moieties of a kind, and present in an amount at least sufficient, to form the HMC reverse micelles having a hydrophobic exterior provided by the hydrophobic moieties and a hydrophilic interior comprised of positively charged chitosan moieties.

6. The method of claim 5 further comprising delivering the HMC reverse micelles to an internal bleed site or its vicinity by applying the HMC reverse micelles to a tissue shielding the bleed site and through which tissue the HMC reverse micelles penetrate to the bleed site or its vicinity.

7. The method of claim 5 further comprising delivering the HMC reverse micelles to an internal bleed site by delivering the HMC reverse micelles parenterally through the tissue shielding the bleed site and directly to the bleed site or its vicinity.

8. The method of claim 5 further comprising delivering the HMC reverse micelles enterally, parenterally, by inhalation or topically.

9. A nasal spray comprising hydrophobically modified chitosan (“HMC”) reverse micelles comprising biocompatible hydrophobic moieties attached to chitosan molecules wherein the chitosan has a molecular weight of not greater than 60,000 Daltons and a degree of deacetylation greater than about 90%.
